(19) United States
(12) Patent Application Publication
(10) Pub. No.: US 2014/0046166 A1
(43) Pub. Date: Feb. 13, 2014

(54) OBJECT INFORMATION ACQUIRING APPARATUS, CONTROL METHOD THEREOF, AND METHOD FOR DETERMINATION OF CONTACT

(71) Applicant: Toshinobu Tokita, Kyoto-shi (JP)

(72) Inventor: Toshinobu Tokita, Kyoto-shi (JP)

(73) Assignee: CANON KABUSHIKI KAISHA, Tokyo (JP)

(21) Appl. No.: 14/113,665

(22) PCT Filed: Apr. 25, 2012
(86) PCT No.: PCT/JP2012/002834

§ 371 Date: Oct. 24, 2013

(30) Foreign Application Priority Data
May 2, 2011 (JP) 2011-102843

(51) Int. Cl. A61B 5/00

(52) U.S. Cl. CPC A61B 5/0095
USPC 600/407

(57) ABSTRACT

The present invention employs an object information acquiring apparatus having: a photoacoustic probe unit including a light irradiating unit which irradiates light, and a probe which receives a photoacoustic wave generated from an object irradiated with light while transmitting an ultrasound wave to the object and receiving a reflected wave thereof; a processor configured to create image information of the object based on the photoacoustic wave; and a controller configured to control irradiation with light, wherein when a first contact condition is defined as a condition in which the object is irradiated with light while the probe is acoustically matched with the object, the controller determines whether or not the photoacoustic probe unit is in the first contact condition by using the reflected wave and enables irradiation with light when the photoacoustic probe unit is in the first contact condition.

### TECHNICAL FIELD

The present invention relates to an object information acquiring apparatus, a control method thereof, and a method for determination of a contact.

### BACKGROUND ART

Attention has been focused on photoacoustic tomography (hereinafter will be referred to as “PAT”) as a method of specifically imaging neovascularization which occurs due to cancer. PAT is a method including illuminating an object with illuminating light such as near infrared rays and receiving a photoacoustic wave generated from the inside of the object by means of an ultrasound probe, thereby imaging the photoacoustic wave. Such a photoacoustic apparatus is described in NPL 1.

NPL 1, however, is silent on a contact between an illuminating light emitting surface and the object. Therefore, it is possible that the illuminating light is emitted not only to the object but also into other space and, hence, there is room to improve the safety against illuminating light.

PTL 1 describes a technique for addressing this problem. FIG. 7 illustrates the configuration of the technique of PTL 1. FIG. 7A is a sectional view and FIG. 7B is a bottom view. In FIG. 7, an energy emitting surface 101 is a surface for contact with skin from which energy, such as light, is emitted. A support structure 102 fixes the energy emitting surface 101 and is housed in a housing 104 with contact sensors 103 intervening therebetween. The contact sensors 103 are each configured to detect a contact between the energy emitting surface 101 and non-illustrated skin and are disposed to circumscribe the energy emitting surface 101. Energy emission is stopped unless the contact between the contact sensors 103 and the skin is detected. By so doing, energy irradiation is conducted only when the energy emitting surface 101 is completely in intimate contact with the skin, thus improving the safety against energy irradiation.

### CITATION LIST

### [Patent Literature]

### [PTL 1]

Japanese Translation of PCT Application No. 2006-525036

### [Non Patent Literature]

### [NPL 1]

S. A. Ermilov et al., Development of laser optoacoustic and ultrasonic imaging system for breast cancer utilizing handheld array probes, Photons Plus Ultrasound: Imaging and Sensing 2009, Proc. of SPIE vol. 7177, 2009.

### SUMMARY OF INVENTION

### Technical Problem

The conventional art, however, involves the following problems.

Since NPL 1 is silent on a contact between an illuminating light emitting surface and the object, it is possible that the illuminating light is emitted into a space other than object. For this reason, the safety against the illuminating light is not sufficient. Therefore, the operator or user has to exercise a considerable care to avoid emission of the illuminating light into such a space.

The technique described in PTL 1 addresses this problem. Specifically, the contact sensors for contact with skin are disposed around the illuminating light emitting surface to perform a control for stopping emission of the illuminating light unless the contact sensors detect a contact. However, the provision of a multiplicity of such contact sensors around the illuminating light emitting surface causes the system configuration to become larger in size. This leads to lowered operability for the operator or user.

The present invention has been made with the foregoing problems in view. An object of the present invention is to downsize the system configuration for photoacoustic tomography, thereby to improve the operability for the operator or user.

### Solution to Problem

The present invention provides an object information acquiring apparatus comprising:

a photoacoustic probe unit including a light irradiating unit which guides light from a light source to an object, and a probe which receives a photoacoustic wave generated from the object irradiated with light by the light irradiating unit while transmitting an ultrasound wave to the object and receiving a reflected wave thereof;

a processor configured to create image information on an internal part of the object based on the photoacoustic wave received by the probe; and

a controller configured to control irradiation with light from the light irradiating unit,

wherein when a first contact condition is defined as a condition in which the object is irradiated with light from the light irradiating unit while the photoacoustic probe unit is in contact with the object in such a manner that the probe is acoustically matched with the object, the controller determines whether or not the photoacoustic probe unit is in the first contact condition by using the reflected wave and enables irradiation with light from the light irradiating unit to be performed when the photoacoustic probe unit is determined as being in the first contact condition.

The present invention also provides a method for controlling an object information acquiring apparatus having: a photoacoustic probe unit including a light irradiating unit which guides light from a light source to an object and a probe which receives a photoacoustic wave generated from the object irradiated with light by the light irradiating unit while transmitting an ultrasound wave to the object and receiving a reflected wave thereof; a processor configured to create image information on an internal part of the object based on the photoacoustic wave received by the probe; and a controller configured to control irradiation with light from the light irradiating unit,

wherein when a first contact condition is defined as a condition in which the object is irradiated with light from the light irradiating unit while the photoacoustic probe unit is in contact with the object in such a manner that the probe is acoustically matched with the object, the method comprises the steps of: causing the controller to determine whether or not the probe is acoustically matched with the object based on the reflected wave; causing the controller to determine that the photoacoustic probe unit is in the first contact condition when the probe is determined as being acoustically matched with the object; and causing the controller to enable irradiation with light from the light irradiating unit to be performed when the photoacoustic probe unit is determined as being in the first contact condition.

The present invention also provides a method for determining a contact condition between a photoacoustic probe unit and an object,

the photoacoustic probe unit including a light irradiating unit which guides light from a light source to the object, and a probe which receives a photoacoustic wave generated from the object irradiated with light by the light irradiating unit while transmitting an ultrasound wave to the object and receiving a reflected wave thereof,

wherein when a first contact condition is defined as a condition in which the object is irradiated with light from the light irradiating unit while the photoacoustic probe unit is in contact with the object in such a manner that the probe is acoustically matched with the object, the method comprises the steps of: causing an information processor to determine whether or not the probe is acoustically matched with the object based on the reflected wave; and causing the information processor to determine that the photoacoustic probe unit is in the first contact condition when the probe is determined as being acoustically matched with the object.

### Advantageous Effects of Invention

According to the present invention, it is possible to downsize the system configuration for photoacoustic tomography, thereby to improve the operability for the operator or user.

Further features of the present invention will become apparent from the following description of exemplary embodiments with reference to the attached drawings.

### BRIEF DESCRIPTION OF DRAWINGS

FIG. 1 is a view illustrating a system configuration according to an embodiment of the present invention.

FIG. 2 is a view illustrating a system configuration according to Embodiment 1 of the present invention.

FIGS. 3A to 3E are views illustrating photoacoustic probe unit according to Embodiment 1 of the present invention.

FIGS. 4A and 4B are charts illustrating control method according to Embodiment 1 of the present invention.

FIGS. 5A to 5D are charts illustrating method for ultrasound determination of contact according to Embodiment 1 of the present invention.

FIGS. 6A and 6B are charts illustrating control method according to Embodiment 2 of the present invention.

FIGS. 7A and 7B are views illustrating the background art.

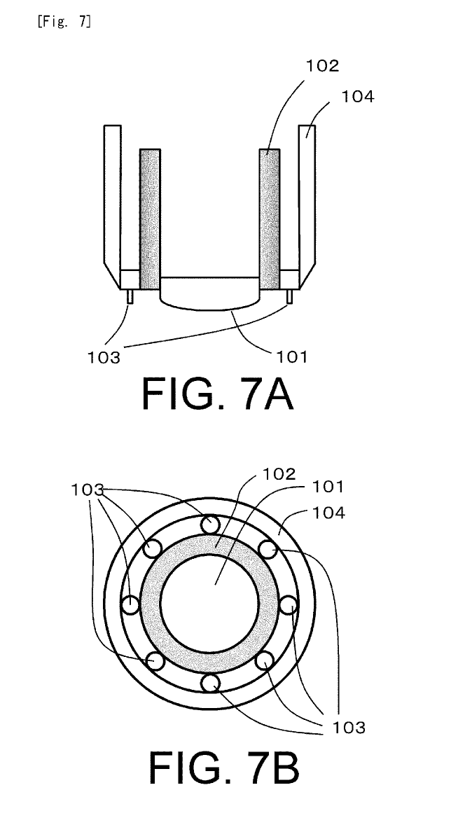

### DESCRIPTION OF EMBODIMENTS

Hereinafter, preferred embodiments of the present invention will be described with reference to the drawings. The present invention is applicable to apparatuses utilizing a photoacoustic effect for acquiring object information as image information by receiving an acoustic wave that is generated in an object by irradiating the object with light (electromagnetic wave). (Such an acoustic wave is also called a “photoacoustic wave”, a typical example of which is an ultrasound wave.) Such apparatuses are called “photoacoustic apparatuses”. A photoacoustic apparatus according to the present invention is configured to utilize the ultrasound echo technique including transmitting an ultrasound wave to the object and then receiving a reflected wave resulting from reflection of the ultrasound wave inside the object to acquire object information as image information. Therefore, the apparatus according to the present invention can be called an object information acquiring apparatus serving both as a photoacoustic apparatus and an ultrasound echo apparatus.

When the apparatus of the present invention is regarded as the former, i.e., the photoacoustic apparatus, the object information to be acquired means a distribution of generation sources of acoustic waves generated by irradiation with light, an initial sound pressure distribution inside the object, an absorbed optical energy density distribution or absorption coefficient distribution derived from the initial sound pressure distribution, or a concentration distribution of a substance forming a tissue. The concentration distribution of a substance is meant to include, for example, an oxygen saturation distribution and an oxidized-reduced hemoglobin concentration distribution.

When the object information acquiring apparatus is regarded as the latter, i.e., the ultrasound echo apparatus, the object information to be acquired is information reflecting an acoustic impedance difference between tissues inside the object.

The “acoustic wave” is typically an ultrasound wave and is meant to include elastic waves such as called a sound wave, an ultrasound wave, an acoustic wave, a photoacoustic wave, and a photoultrasound wave. The “light”, as used in the present invention, is meant by electromagnetic waves including visible rays and infrared rays. Light of a specific wavelength is simply selected to meet a component to be measured by the object information acquiring apparatus.

The following description is directed to the object, though the object does not form part of the object information acquiring apparatus of the present invention. The object information acquiring apparatus according to the present invention is capable of diagnosing malignant tumors, vascular diseases, blood sugar levels, and the like of humans or animals, making follow-up of chemotherapy, and the like. Therefore, the object is assumed as a living body, specifically, a breast, finger, limb or the like of a human or animal. A light absorbing substance present inside the object is a substance having a relatively high absorption coefficient among substances present inside the object. For example, in cases where a human body is a subject of measurement, such light absorbing substances include oxidized or reduced hemoglobin and a blood vessel containing such hemoglobin, and a malignant tumor containing a number of angiogenesis.

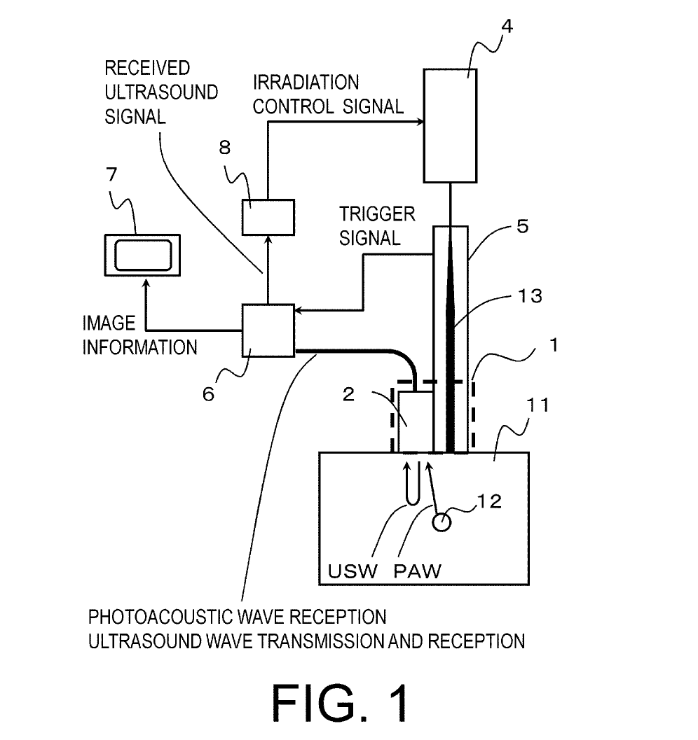

FIG. 1 schematically illustrates a photoacoustic apparatus. A light source 4 emits illuminating light 13, and an illuminating optical system 5 irradiates an object 11 with the illuminating light 13. An irradiating end of the illuminating optical system is located adjacent to a probe 2, and the irradiating end and the probe form a photoacoustic probe unit 1. The illuminating light 13 permeates into the object and then a light absorbing substance 12 present inside the object generates a photoacoustic wave (PAW), which in turn is received by the probe 2. A processing device 6 performs amplification, digital conversion, detection and like processing of a photoacoustic signal received by the probe 2 in synchronization with an illuminating light emission trigger signal to create image information and causes a monitor 7 to display the image information. The processing device is equivalent to the “processor” defined by the present invention.

The probe 2 transmits and receives an ultrasound wave (USW) that is beam formed by the processing device 6 to and from the object 11 in order to acquire an ultrasound image. When the probe cannot receive any reflected wave from the object 11, a control device 8 determines that the photoacoustic probe unit 1 is out of contact with the object 11. When the probe has received a reflected wave from the object 11, on the other hand, the control device 8 determines that the photoacoustic probe unit 1 is in contact with the object 11. In this way, the control device determines the condition of contact with the object 11. The processing device 6 performs an irradiation control for causing the condition of contact with the object to be determined in the above-described manner and permitting irradiation during a contact condition while inhibiting irradiation during a non-contact condition.

The configuration described above makes it possible to downsize the photoacoustic probe unit 1 because the probe 2, which is a constituent element of the photoacoustic apparatus, can be utilized as a contact sensor. This leads to a simplified system configuration.

### Embodiment 1

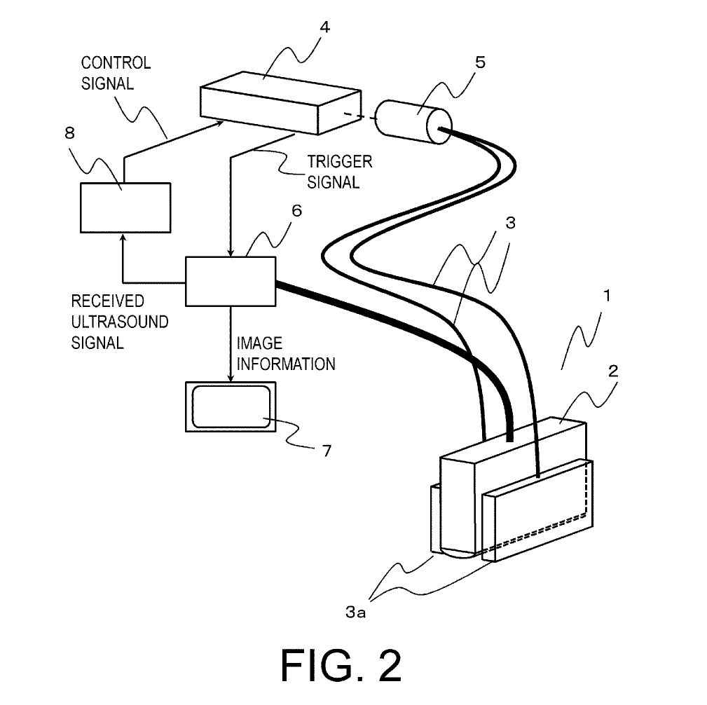

In Embodiment 1, the photoacoustic apparatus is described more specifically with reference to FIG. 2. The photoacoustic probe unit 1 comprises the probe 2 configured to receive a photoacoustic wave generated from the object (not shown) and the irradiating end for irradiating the object with illuminating light comprising near infrared rays. In one example, the irradiating end is provided with a bundled fiber 3. Though FIG. 1 does not show an illuminating optical system for guiding illuminating light from an irradiating end 3a of the bundled fiber to the object, it is possible to irradiate the object directly from the irradiating end 3a of the bundled fiber 3 or to provide any desired optical element such as a lens or a diffuser. The irradiating end is equivalent to the “light irradiating unit” defined by the present invention.

The light source 4 generates light (which comprises near infrared rays according to the present embodiment). The illuminating optical system 5 is configured to form the generated light into a beam having a beam radius and causes the light to become incident on the bundled fiber 3. A pulse laser, such as an Nd:YAG laser or an alexandrite laser, is used as the light source 1. Alternatively, use may be made of a Ti:Sa laser or OPO laser which uses Nd: YAG laser light as excitation light.

The illuminating light is partially branched to measure light emission of the light source 1 by means of a photodiode (not shown). The output of the photodiode is used as a trigger signal. When the trigger signal is inputted to the processing device 6, the probe 2 acquires a photoacoustic signal. Thereafter, the photoacoustic signal is subjected to amplification, digital conversion, detection and the like to create image information, which in turn is displayed on the monitor 7. The trigger signal is not limited to a signal generated by the photodiode, but it is effective to use a method of synchronizing light emission of the light source 4 to an input trigger for the processing device 6 by using a signal generator.

The probe 2 transmits and receives an ultrasound wave which is beam formed by the processing device 6 to and from the object in order to acquire an ultrasound image. The ultrasound wave is used not only in acquiring the ultrasound image but also in determining a contact between the photoacoustic probe unit 1 and the object are in contact with each other. An array of detecting elements such as PZTs or CMUTs, for example, can be used as the probe 2.

When the reflected wave from the object cannot be received, the control device 8 determines that the photoacoustic probe unit 1 is out of contact with the object. When the reflected wave from the object has been received, on the other hand, the control device determines that the photoacoustic probe unit 1 is in contact with the object. In this way, the control device 8 determines the condition of contact with the object and transmits an irradiation control signal to the light source 1. The control device is equivalent to the “controller” defined by the present invention. The processing device and the control device can be implemented by using an information processor for example. The information processing device may perform different processing operations by using respective circuits dedicated thereto or may be implemented in the form of a program for operating a computer having a CPU or the like.

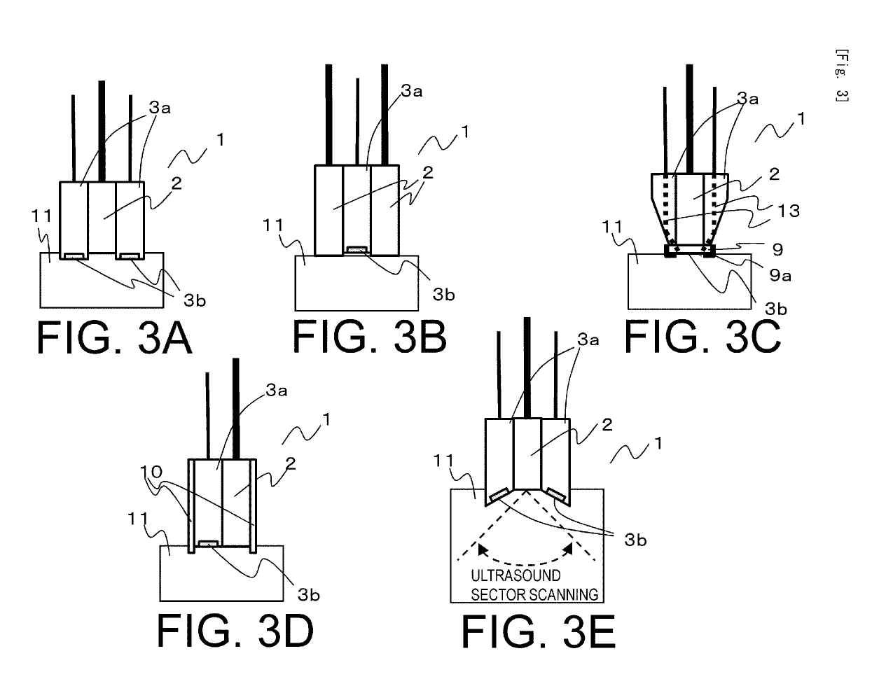

With reference to FIG. 3, description is made below of the configuration of the photoacoustic probe unit 1.

When an ultrasound wave transmitting and receiving portion of the probe 2 is acoustically matched with the object 11, the irradiating end of the photoacoustic probe unit 1 for emitting illuminating light 13 needs to be in contact with the object. Alternatively, another member located around the irradiating end needs to be in contact with the object. By so doing, it becomes possible to considerably reduce the illuminating light 13 emitted from between the photoacoustic probe unit 1 and the object 11. With reference to FIGS. 3A to 3E, different configurations for the respective cases are described below.

The photoacoustic probe unit 1 shown in FIG. 3A has a configuration in which the probe 2 is sandwiched between irradiating ends 3a of the bundled fiber from outside in such a manner that the ultrasound wave transmitting and receiving surface of the probe 2 is positioned more apart from the object 11 than irradiating ends 3b. Alternatively, the probe 2 may be circumscribed by the irradiating ends 3a from outside. By thus providing a difference in height between the probe 2 and the illuminating light irradiating ends 3b, the illuminating light emitting ends 3b are brought into contact with the object 11 as long as the ultrasound wave transmitting and receiving surface of the probe is in contact with the object 11.

The photoacoustic probe unit 1 shown in FIG. 3B has a configuration in which an irradiating end 3a of the bundled fiber is sandwiched between probes 2 from outside in such a manner that an irradiating end 3a of the bundled fiber is positioned coplanar with the probes 2 with respect to the object 11 or more apart from the object than the probes. Alternatively, the irradiating end 3a may be surrounded by the probes 2 from outside. By thus providing a difference in height between the probes 2 and the illuminating light irradiating end 3b, the illuminating light irradiating end 3b or the probes around the irradiating end are brought into contact with the object as long as the ultrasound wave transmitting and receiving surfaces of the probes are in contact with the object 11. That is, the object is irradiated with light from the irradiating end of the photoacoustic probe unit, while the probe is acoustically matched with the object. The contact condition assumed at that time is a first contact condition which is an adequate contact condition.

The photoacoustic probe unit 1 shown in FIG. 3C has a configuration in which the ultrasound wave transmitting and receiving surface of the probe 2 is provided with an acoustically matching member 9 so that the illuminating light 13 emitted from irradiating ends 3a of the bundled fiber passes toward the irradiating end 3b through the acoustically matching member 9 to illuminate the object 11. Preferably, side surfaces of the acoustically matching member 9 and that surface of the acoustically matching member which faces the object except the irradiating end 3b are provided with a light-shielding material 9a (i.e., reflecting material or absorbing material) for blocking the illuminating light 13 in order to prevent the illuminating light 13 from emitting from a portion other than the emitting end 3b. The acoustically matching member 9 comprises a material having a high transmissive property with respect to sound and illuminating light 13, preferred examples of which include polymethylpentene and urethane. The acoustically matching member 9 may serve to diffuse the illuminating light 13. By thus providing a difference in height between the ultrasound wave transmitting and receiving surface of the probe 2 and the illuminating light irradiating end 3b with respect to the object 11, the illuminating light irradiating end 3b is brought into contact with the object 11 when the probe 2 is acoustically matched with the object 11 by means of the acoustically matching member 9.

The photoacoustic probe unit 1 shown in FIG. 3D has a configuration in which both the probe 2 and the irradiating end 3a are sandwiched by a light-shielding walls 10 from outside in such a manner as to provide unevenness on that surface of the photoacoustic probe unit 1 which faces the object, though the ultrasound wave transmitting and receiving surface of the probe 2 and the illuminating light emitting end 3b are of the same height. Alternatively, the photoacoustic probe unit may be wholly surrounded by the light-shielding wall 10 from outside. The light-shielding wall is positioned so as not to interfere with ultrasound wave transmission and reception and irradiation with light. This feature allows the illuminating light irradiating end 3b or the light-shielding wall 10 around the irradiating end 3b to be brought into contact with the object 11 as long as the ultrasound wave transmitting and receiving surface of the probe 2 is in contact with the object 11.

As a variation of the photoacoustic probe unit 1 shown in FIG. 3A, an irradiating end 3b may be tapered or provided with a curvature, as shown in FIG. 3E. This feature allows the object 11 to be easily fitted into the recess defined between the tapered portions of the irradiating end 3b. Therefore, the illuminating light irradiating end 3b is brought into contact with the object 11 more reliably as long as the ultrasound wave transmitting and receiving surface of the probe 2 is in contact with the object 11. Use of probe 2 capable of sector scanning enables ultrasound wave transmission and reception by the probe 2 for determination of contact with the object 11 to be performed in a sector scanning manner without interference with the irradiating end 3b, thus making it possible to realize determination of contact with a broader area.

Though in all the configurations shown in FIGS. 3A to 3E the surface for contact with the object has corners each right-angled or acute-angled, such corners are preferably rounded for actual use.

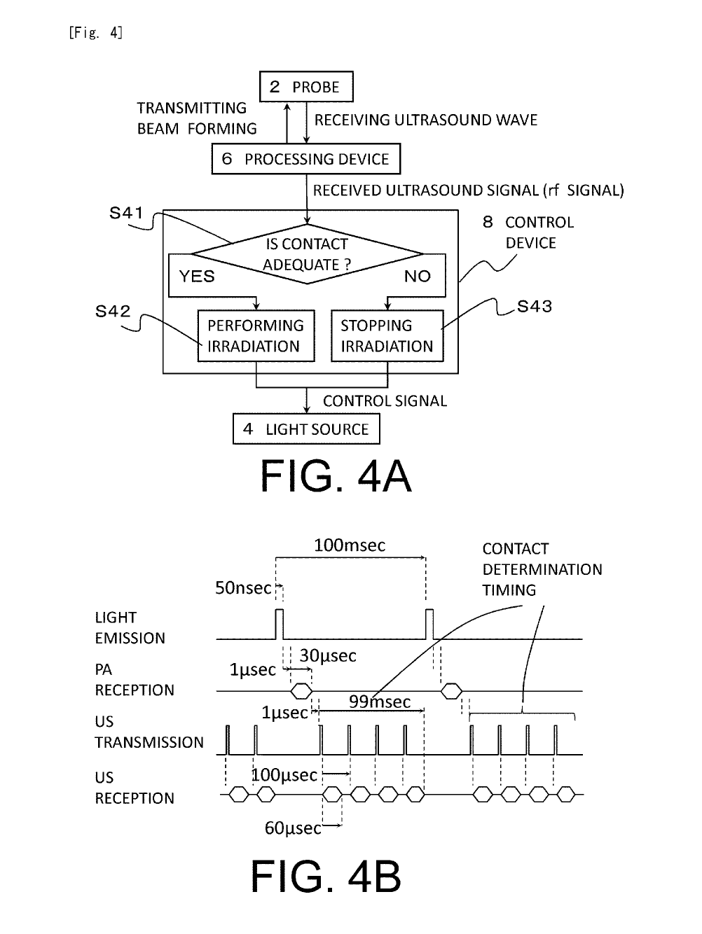

With reference to FIG. 4A, description is made below of a control method carried out by the control device 8.

FIG. 4A is a flowchart illustrating an irradiation control performed by the control device 8. The processing device 6 performs transmit beam forming on a vibrator in the probe 2 for acquiring an ultrasound image. Then, the processing device 6 performs reception beam forming on a signal received by the probe 2 from the object to acquire the ultrasound image. On the other hand, the processing device 6 transmits a received ultrasound signal (rf signal) to the control device 8.

In turn, the control device 8 starts a control process. Initially, the control device determines from the rf signal whether or not the contact between the photoacoustic probe unit 1 and the object is adequate (step S41).

If the contact condition is determined as being adequate (S41=YES), the control device permits irradiation (step S42).

If the contact condition is determined as being inadequate (S41=NO), on the other hand, the control device transmits an irradiation control signal to the light source 4 to stop irradiation with the illuminating light (step S43).

Either a method including opening and closing an internal shutter of the light source 4 or a method including controlling an internal trigger signal (generated by a flash lamp and a Q switch) is effective for the irradiation control over the light source 4. While the control signal from the control device 8 has been described as a signal for the irradiation control over the light source 1, there is no limitation to this feature. For example, it is possible that an external shutter is provided between the light source 1 and the illuminating optical system 2 while the opening and closing of the shutter is controlled by such a control signal.

With reference to the timing chart of FIG. 4B, description of irradiation control timing is made below. In FIG. 4B, photoacoustic signal reception (PA reception) is to acquire signals for a predetermined time period (e.g., 30 microseconds) in response to light emission serving as a trigger. The light emission interval is determined from the emission frequency of the light source 4. When the emission frequency is 10 Hz for example, the light emission interval is 100 msec. That is, the time period from the end of photoacoustic signal acquisition to the next light emission is not less than 99 msec. During this time period, the irradiation control described using the flowchart of FIG. 4A is performed. When the irradiation is stopped because of an inadequate contact, the irradiation is suspended until a contact is made. Immediately after the contact has been made, the irradiation control signal is transmitted to the light source 4 for causing the light source to perform irradiation. In cases where the light source 4 is controlled by the flash lamp and the Q switch, irradiation is performed at the next light emission timing in synchronization with the flash lamp.

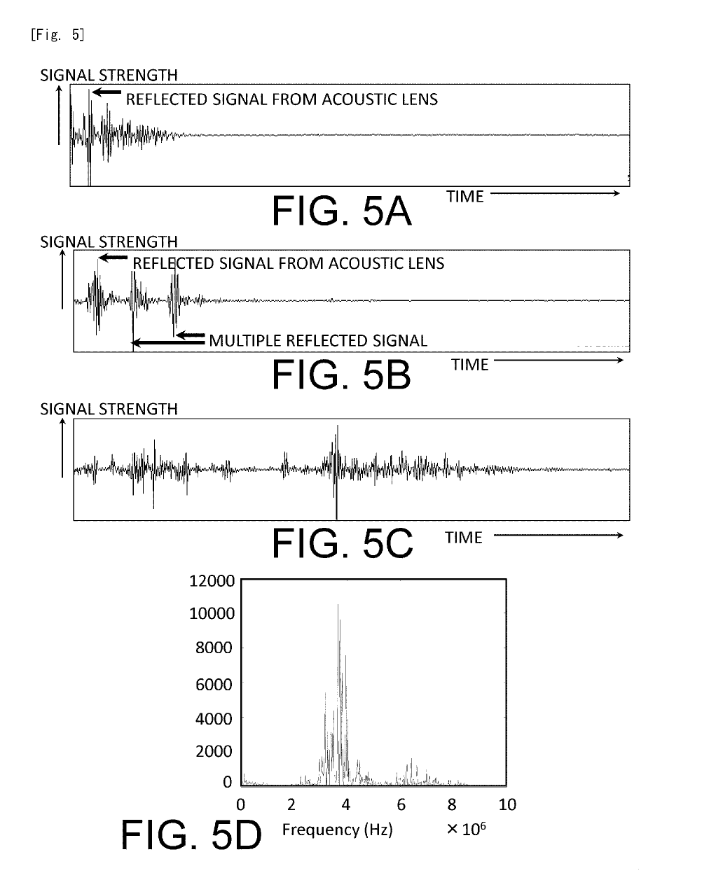

With reference to FIG. 5, description is made below of determination of contact between the photoacoustic probe unit 1 and the object by means of the probe 2.

FIG. 5 shows a waveform received by the probe 2 (rf signal resulting after delay and sum). FIG. 5A shows a waveform received when the probe 2 is not in contact with any matter. FIG. 5B is a waveform received when only sonar gel is attached to the surface of the probe 2. FIG. 5C shows a waveform received when the surface of the probe 2 is in contact with the object via the sonar gel. The sonar gel functions as an acoustically matching agent and forms a matching layer. Further, the probe is in contact with an acoustic lens which functions as an acoustic member in receiving an ultrasound wave.

In FIGS. 5A and 5B, signals are detected from a range down to a predetermined depth which is coincident with or adjacent to the boundary between the acoustic lens and air, though the probe is not in contact with any matter actually (i.e., in air). Specifically, reflected signals from a range from the vibrator in the probe 2 down to the acoustic lens are observed in FIG. 5A. In FIG. 5B, in addition to the reflected signals from the range down to the acoustic lens, multiple reflected signals from the acoustic lens and from the matching layer are also observed. From a position deeper than the predetermined depth, only noise-level signals of the probe 2 and processing device 6 are observed. Therefore, in the cases of FIGS. 5A and 5B, it can be determined that no reflected signals are acquired from the object.

In FIG. 5C, on the other hand, such reflected signals (from positions coincident with the acoustic lens and the matching layer) are relatively small as compared with FIGS. 5A and 5B. Further, ultrasound signals are received from a deeper position than the above-described predetermined depth (from which reflected waves are received later). Therefore, it can be determined that in the case of FIG. 5C an ultrasound wave has permeated into a substance having a relatively small acoustic impedance difference (e.g., the object in a state of being acoustically matched with the probe), whereas in the cases of FIGS. 5A and 5B the acoustic impedance has varied largely because of an ultrasound wave permeating into air.

In this way, in the case where signals from the predetermined depth which is coincident with or adjacent to the boundary of the photoacoustic probe unit are present and larger than signals from the object acoustically matched with the photoacoustic probe unit or multiple reflection takes place, it can be determined that the photoacoustic probe unit 1 is out of contact with the object. In the case where there are no signals or small signals from the predetermined depth, it can be determined that the photoacoustic probe unit is in contact with the object.

Whether or not signals from the predetermined depth are present can be determined by determining whether or not an rf signal is acquired which is several times as large as the S/N ratio of the probe 2 and processing device 6. For example, if there is a signal from a depth of 10 mm which is twice or more times, preferably three or more times as large as the S/N ratio, the probe 2 can be determined as being in contact with the object. There is no limitation to the depth of 10 mm. It is preferable to determine signals from plural points. There is no limitation to the value of twice or more times or three or more times as large as the S/N ratio which is used as a criterion for determination.

The presence or absence of a reflected signal from the range from the vibrator in the probe 2 to the matching layer or the acoustic lens can be determined from the presence or absence of a repeated signal within the range down to the predetermined depth on which attention is focused here. For example, the presence or absence of such a reflected signal can be determined by conducting Fourier transform of an rf signal from the range down to a depth of 5 mm and determining the presence or absence of a predetermined frequency component. Assuming that the average acoustic velocity from the vibrator of the probe 2 to the acoustic lens is 2000 m/s and the thickness is 0.25 mm, a frequency component corresponding to a round-trip distance of 0.5 mm which is an ultrasound propagation distance is detected (200 m/s/0.5 mm=4 MHz). This can be seen from a peak of the rf signal having been subjected to Fourier transform which appears at around 3.8 MHz as shown in FIG. 5D resulting from Fourier transform of FIG. 5A.

It is needless to say that the depth specified here varies depending on the structure of the photoacoustic probe unit, the thickness of the object and the like and has to be appropriately established to meet such conditions.

The frequency component to be determined may be selected from a range, for example, a range from 3 MHz to 4 MHz in the example shown in FIG. 5D. Since the frequency specified here is attributable to the structure of the probe, there is no limitation to this value. Use may be made of any other method of detecting multiple reflected signals.

Alternatively, it is possible to determine that the probe 2 and the object are out of contact with each other when only the noise components inherent in the probe 2 and processing device 6 are detected from rf signals from a depth of not less than 7 mm for example which have been subjected to Fourier transform. When a frequency component other than those corresponding to noises is detected, the probe 2 and the object may be determined as being in contact with each other.

As described above, by conducting determination using at least one of a signal from the predetermined depth and a reflected signal from the range from the vibrator in the probe 2 to the matching layer or the acoustic lens, determination of contact between the probe 2 and the object becomes possible. As described with reference to FIG. 3, when the probe 2 of the photoacoustic probe unit 1 is acoustically matched with the object, the illuminating light irradiating end or another member located around the irradiating end is in contact with the object. When the illuminating light is emitted against the object from the photoacoustic probe unit 1 assuming such a condition, it becomes possible to considerably reduce the illuminating light emitted from between the photoacoustic probe unit 1 and the object.

While the determination of contact using rf signals obtained after having been subjected to phasing and adding has been described herein, a similar method may be applied to ultrasound wave transmission and reception relying upon a single element. Likewise, a similar method may be applied to signals obtained after having been subjected to detection. Though the determination of contact is preferably conducted based on all the received ultrasound signals (rf signals), the determination of contact is possible based on at least ultrasound signals received at opposite ends or corners of the probe 2 (particularly in the case of a two-dimensional array probe) is possible.

While the method having been described carries out ultrasound wave transmission and reception for both of the ultrasound image acquisition and the determination of contact, ultrasound wave transmission and reception may be carried out only for the determination of contact without acquiring an ultrasound image. In such a case, transmit and reception beam forming is unnecessary and the determination of contact between the photoacoustic probe unit 1 and the object can be carried out based on received signals acquired by driving a plurality of vibrators in the probe 2.

As described above, the probe 2, which is a constituent element of the photoacoustic apparatus, can be utilized as a contact sensor and, for this reason, the photoacoustic probe unit 1 can be downsized. When the number of ultrasound transmission and reception beams used in the determination of contact is increased, multiple determination becomes possible, which leads to improved safety. Further, since the determination of contact and the ultrasound image acquisition can be performed at the same time, illuminating light irradiation control and data acquisition can be performed efficiently.

In the present embodiment, description has been made of the configuration utilizing ultrasound wave transmission and reception by the probe 2 serving as one of the contact sensors and the control method thereof. It is natural that a separate contact sensor may be provided for multiple safety measures. In contrast to multiple safety measures using plural types of contact sensors without the probe 2, the present embodiment can use the probe 2 as one contact sensor and hence can realize downsizing of the whole photoacoustic probe unit 1.

### Embodiment 2

In Embodiment 1, description has been made of the determination of contact between the photoacoustic probe unit 1 and the object carried out by transmitting and receiving ultrasound waves to and from the object by means of the probe 2, as well as the irradiation control. In Embodiment 2, description is made of a method of irradiation control by foreseeing the photoacoustic probe unit 1 in a state of being about to separate from the object based on the result of repeated ultrasound wave transmission and reception.

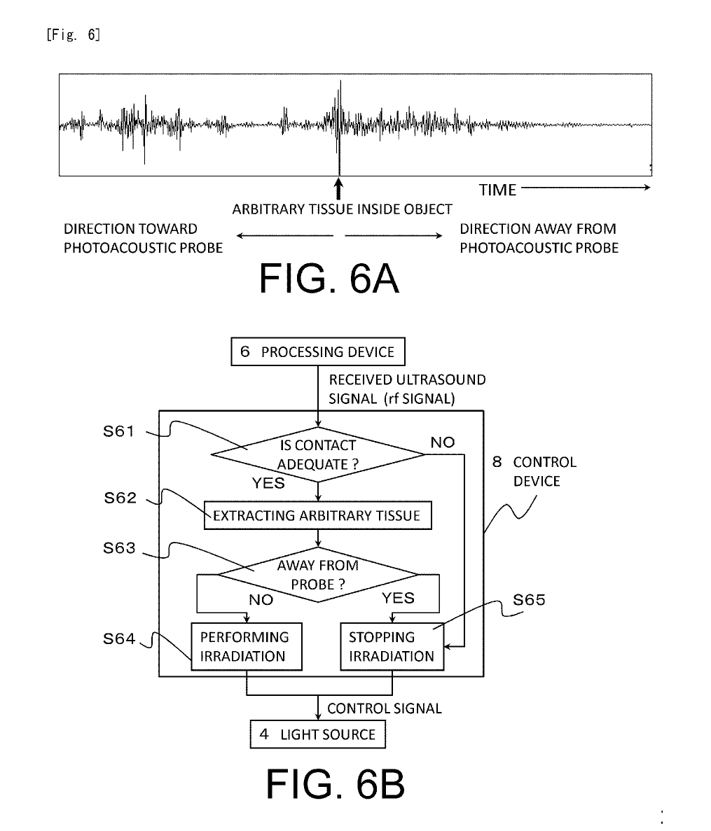

Like FIG. 5C, FIG. 6A shows an ultrasound signal (rf signal) received when the probe 2 is in contact with the object. As indicated by arrow in FIG. 6A, an rf signal from an arbitrary tissue inside the object is observed.

The irradiation control is described below with reference to the flowchart of FIG. 6B.

According to the process for determination of contact described in Embodiment 1, the control device 8 determines from the rf signal whether or not the contact between the photoacoustic probe unit 1 and the object is adequate (step S61).

If the contact is determined as being adequate (S61=YES), the control device extracts an rf signal from an arbitrary tissue inside the object (step S62). Preferably, the “arbitrary tissue” is a tissue expansively present in the object like a subcutaneous fat layer for example. If the contact is determined as being inadequate (S61=NO), the control device stops irradiation (step S65).

Subsequent to step S62, the control device determines a movement of the tissue relative to the photoacoustic probe unit 1. Thereafter, the control device determines whether or not the movement of the tissue is in the direction away from the photoacoustic probe unit (step S63).

If the time required for transmission and reception becomes shorter or remains the same, the tissue is considered as moving in the direction toward the photoacoustic probe unit indicated in FIG. 6A or remaining stationary. That is, the control device does not determine that the tissue is moving in the direction away from the photoacoustic probe unit (S63=NO) and then permits irradiation with illuminating light (step S64).

On the other hand, if the time required for transmission and reception becomes longer, i.e., if the tissue is moving in the direction away from the photoacoustic probe unit indicated in FIG. 6A, the control device foresees that the photoacoustic probe unit 1 will separate from the object soon (S63=YES) and then performs an irradiation control for stopping irradiation in advance (step S65). Ina more preferable arrangement, a temporal differential of the amount of the movement, i.e., a threshold value of the velocity of the movement, is provided and an irradiation control for stopping irradiation is performed when the velocity exceeds the threshold value.

As described above, the present embodiment is capable of performing irradiation control by foreseeing the contact condition between the photoacoustic probe unit 1 and the object and hence further improves the safety.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-102843, filed on May 2, 2011, which is hereby incorporated by reference herein in its entirety.

1. An object information acquiring apparatus comprising:
a photoacoustic probe unit including a light irradiating unit which directs light from a light source to an object, and a probe which receives a photoacoustic wave generated from the object irradiated with light by said light irradiating unit while transmitting an ultrasound wave to the object and receiving a reflected wave thereof;
a processor configured to create image information on an internal part of the object based on the photoacoustic wave received by said probe; and
a controller configured to control irradiation with light from said light irradiating unit,
wherein, when a first contact condition is defined as a condition in which the object is irradiated with light from said light irradiating unit while said photoacoustic probe unit is in contact with the object in such a manner that said probe is acoustically matched with the object, said controller determines whether or not said photoacoustic probe unit is in the first contact condition by using the reflected wave and enables irradiation with light from said light irradiating unit to be performed when said photoacoustic probe unit is determined as being in the first contact condition.

2. The object information acquiring apparatus according to claim 1,
wherein said light irradiating unit is located outwardly relative to said probe, and
wherein said probe has an ultrasound wave transmitting and receiving surface positioned farther from the object than is said light irradiating unit.

3. The object information acquiring apparatus according to claim 1,
wherein said probe is located outward relative to said light irradiating unit, and
wherein said probe has an ultrasound wave transmitting and receiving surface, and said light irradiating unit has an irradiating end which is positioned coplanar with said ultrasound wave transmitting and receiving surface of said probe with respect to the object or positioned farther from the object than is said ultrasound wave transmitting and receiving surface of said probe.

4. The object information acquiring apparatus according to claim 1, wherein said probe has an ultrasound wave transmitting and receiving surface, and wherein said ultrasound wave transmitting and receiving surface of said probe is provided with an acoustic matching agent, and wherein an irradiating end of said light irradiating unit is in contact with said acoustic matching agent.

5. The object information acquiring apparatus according to claim 1, wherein said photoacoustic probe unit further includes a light-shielding wall located outward from said probe and said light irradiating unit, and has an ultrasound wave transmitting and receiving surface, and wherein said uswt of said probe and an irradiating end of said light irradiating unit are positioned farther from the object than is an end of said light-shielding wall.

6. The object information acquiring apparatus according to claim 1, wherein said controller determines whether or not said probe is acoustically matched with the object based on the reflected wave and then determines that said photoacoustic probe unit is in the first contact condition when said probe is determined as being acoustically matched with the object.

7. The object information acquiring apparatus according to claim 6, wherein said controller determines that said probe is not acoustically matched with the object when the reflected wave acquired from a position coinciding with a boundary between said photoacoustic probe unit and the object is larger than a signal acquired from the object in a state of being acoustically matched.

8. The object information acquiring apparatus according to claim 6, wherein said controller determines that said probe is acoustically matched with the object when the reflected wave acquired from a position coinciding with an internal part of the object is larger than noises generated from said probe and said processor.

9. The object information acquiring apparatus according to claim 6, wherein said controller determines that said probe is acoustically matched with the object when the reflected wave acquired from a position coinciding with an internal part of the object contains a frequency component other than frequency components corresponding to noises generated from said probe and said processor.

10. The object information acquiring apparatus according to claim 1, wherein said controller determines whether or not said photoacoustic probe unit in the first contact condition is moving in a direction away from the object, and stops irradiation with light from said light irradiating unit when said photoacoustic probe unit is determined as moving in the direction away from the object.

11. The object information acquiring apparatus according to claim 10,
wherein said probe transmits the ultrasound wave repeatedly to the object and receives the reflected wave thereof, and
wherein said controller extracts a signal corresponding to a tissue inside the object from the reflected wave, and then determines that said photoacoustic probe unit is moving in the direction away from the object when a longer time is required for later ultrasound wave transmission and reception to acquire the signal thus extracted.

12. A method for controlling an object information acquiring apparatus having a photoacoustic probe unit including a light irradiating unit which directs light from a light source to an object and a probe which receives a photoacoustic wave generated from the object irradiated with light provided by the light irradiating unit while transmitting an ultrasound wave to the object and receiving a reflected wave thereof, a processor configured to create image information on an internal part of the object based on the photoacoustic wave received by the probe, and a controller configured to control irradiation with light from the light irradiating unit, the method comprising the steps of:
causing the controller to determine whether or not the probe is acoustically matched with the object based on the reflected wave;
causing the controller to determine that the photoacoustic probe unit is in a first contact condition when the probe is determined as being acoustically matched with the object, wherein the first contact condition is defined as a condition in which the object is irradiated with light from the light irradiating unit while the photoacoustic probe unit is in contact with the object in such a manner that the probe is acoustically matched with the object; and
causing the controller to enable irradiation with light from the light irradiating unit to be performed when the photoacoustic probe unit is determined as being in the first contact condition.

13. (canceled)
